(19) United States
(12) Patent Application Publication
(10) Pub. No.: US 2013/0267735 A1
(43) Pub. Date: Oct. 10, 2013

(54) Propane Oxidation Process Using Reduced Amounts of Steam

(71) Applicants: Scott Han, Lawrenceville, NJ (US); Christoher Frick, Pottstown, PA (US); Daniel J. Martenak, Perkasie, PA (US); Nelson Quiros, Houston, TX (US)

(72) Inventors: Scott Han, Lawrenceville, NJ (US); Christoher Frick, Pottstown, PA (US); Daniel J. Martenak, Perkasie, PA (US); Nelson Quiros, Houston, TX (US)

(21) Appl. No.: 13/991,826

(22) PCT Filed: Dec. 7, 2011
(86) PCT No.: PCT/US2011/063687

§ 371 Date: Jun. 5, 2013

Related U.S. Application Data
(60) Provisional application No. 61/428,086, filed on Dec. 29, 2010.

(51) Int. Cl. C07C 51/215

(52) U.S. Cl. CPC C07C 51/215
USPC 562/549

(57) ABSTRACT

Less propionic acid is produced as a by-product in a process for the direct oxidation of propane to acrylic acid, the process comprising the step of contacting under oxidation conditions a gaseous mixture comprising (i) propane, (ii) oxygen, (iii) steam and (iv) a diluent gas, with a propane oxidation catalyst by reducing the steam content of the gaseous mixture.

### BACKGROUND OF THE INVENTION

1. Field of the Invention

This invention relates to propane oxidation. In one aspect the invention relates to an improved process for propane oxidation while in another aspect, the invention relates to a propane oxidation process using a reduced amount of steam.

2. Description of the Related Art

In the direct oxidation of propane to acrylic acid (AA) process, propionic acid (PA) is formed as an undesirable side product. In one embodiment of the propane to AA process, the preferred catalyst system consists of mixed metal oxides of molybdenum, vanadium, tellurium and niobium (Mo/V/Te/Nb). These catalysts produce PA at levels that may range from over 1,000 parts per million (ppm) to less than 10,000 ppm when operating under conditions to achieve maximum AA yield (equal to or greater than (≧) 85% oxygen conversion).

In addition, excess propionic acid byproduct can, upon esterification of the AA product, impart undesirable characteristics such as high volatile organic content (VOC), odor or color to the acrylate ester (AE) and its corresponding polymer products. Typically, PA specifications for AA product streams from conventional propylene oxidation, prior to esterification, range from 500-1000 ppm, well below the levels seen in the propane oxidation product. Thus, in order for propane oxidation to be more economically desirable, PA byproduct levels need to be reduced either through the oxidation step or in downstream separation steps.

The separation of propionic acid from acrylic acid is problematic. The boiling points of both are less than (<) 1° C. apart, and they are not capable of separation by distillation. Although methods exist for PA separation, e.g., melt crystallization, these steps substantially increase the capital and operating cost of the AA purification process by requiring additional equipment and utilities to effect the desired separation. Thus it would be advantageous and desirable to be able to control the AA process in such a way as to reduce the formation of PA such that additional costly purification is substantially reduced.

### SUMMARY OF THE INVENTION

In one embodiment of the invention, a propane oxidation process uses a reduced amount of steam to reduce the amount of PA in the process. Typically the process for oxidizing propane to acrylic acid comprises the step of contacting under oxidation conditions propane, oxygen, steam and, optionally, a diluent gas, e.g., nitrogen, with a propane oxidation catalyst. As steam is necessary for the economical conversion of propane to AA, it is not intuitively obvious that it may be advantageous to significantly reduce the amount of steam in the process. Surprisingly, however, significant levels of PA are reduced when steam feed concentrations are lowered. Some corresponding AA yield is lost, but the economic benefit derived from reducing the capital and operating costs of the process required to separate PA from the final product can offset the yield loss. This process can also be used to reduce PA levels in products from conventional propylene or propane-containing propylene streams.

In one embodiment the invention is a process for the direct oxidation of propane to acrylic acid, the process comprising the step of contacting under oxidation conditions a gaseous mixture comprising, in weight percent (wt %) based on the weight of the mixture, 5-10% propane, 5-15% oxygen, 1-20% (preferably 10-15%) steam and the balance a diluent gas, with a propane oxidation catalyst. The amount of PA produced is typically less than 1,000, or less than 750, or less than 500, ppm.

### DETAILED DESCRIPTION OF THE PREFERRED EMBODIMENT

### Definitions

Unless stated to the contrary, implicit from the context, or customary in the art, all parts and percents are based on weight and all test methods are current as of the filing date of this disclosure. For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, etc., is from 100 to 1,000, then all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, the oxidation conditions of the process, PA content in AA streams, and the like.

Process Reactants

The starting materials are generally propane gas, at least one oxygen-containing gas, steam and a diluent gas. The propane does not have to meet any particularly high purity standard, and it may contain propylene or other hydrocarbons or heteroatom-containing hydrocarbons as impurities. In one embodiment the propane does not contain any appreciable amount of propylene, e.g., less than 1, or less than 0.5, or less than 0.1, wt % propylene based on the weight of the propane. In one embodiment the propane contains a relatively large amount, e.g., 1 or more wt %, of propene such as that found in lower-grade propane feeds such as those from fluid catalytic crackers.

The oxygen-containing gases used in the practice of this invention may be pure oxygen gas, an oxygen-containing gas such as air, an oxygen-enriched gas, or a mixture comprising two or more of these gases. The diluent gas is typically an inert gas such as but not limited to nitrogen, argon, helium, and carbon dioxide. The diluting gas may be used to dilute the starting material and/or to adjust the space velocity, the oxygen partial pressure, and the steam partial pressure. Each of these gases may be added to the process individually or in combination with one or more of the other gases.

In one embodiment the propane can be supplemented or replaced with another alkane suitable for gas phase oxidation into an unsaturated aldehyde or carboxylic acid. Generally, the alkane other than propane is a C₄₋₈ alkane, typically isobutane or n-butane. Like propane, these other alkanes do not have to meet any particularly high purity standard, and these may contain one or more C₃₋₈ alkenes as an impurity. Typical alkenes include propene, isobutene, n-butene, pentene, and the like.

In one embodiment a C₃₋₈ alkene feed replaces the propane, and this alkene feed may contain a significant amount of alkane, e.g. up to 49 weight percent (wt %). In one embodiment the feed is isobutene.

Suitable molar ratios of the propane/oxygen/diluting gas/water in the starting material gas mixture are known in the art as well as the feed ratio of propane/air/steam. For instance suitable ranges are disclosed in U.S. Pat. No. 5,380,933. Typical ranges include propane to oxygen to water to diluent of 1:(0.1-10):(0-50):(0-50), more typically 1:(0.5-5):(1-30):(0-30). In one embodiment the starting gas mixture comprises from 5 to 10, or from 6 to 8, weight percent (wt %) propane; from 10 to 20 wt % oxygen; from 1 to 50 wt % steam; and the balance nitrogen.

Process Conditions

The starting gas mixture is subjected to oxidation with an oxidation catalyst. The reaction is generally conducted under atmospheric pressure, but may be conducted under elevated or reduced pressure. Typically the reaction pressure is from 0 to 100, more typically from 0 to 50 pounds per square inch gauge (psig) (0 to 0.70 MegaPascals (MPa), more typically 0 to 0.35 MPa). The reaction temperature is generally from 0 to 550° C., more typically from 200 to 500° C., even more typically from 300 to 480° C. and even more typically from 350 to 440° C. The gas space velocity is generally 100 to 10,000 hr⁻¹, more typically 300 to 6,000 hr⁻¹ and even more typically 300 to 3,000 hr⁻. Residence time of the starting gas mixture in the reactor is typically from 0.1 to 10 seconds, more typically from 1 to 4 seconds.

Oxidation Catalyst

The oxidation catalysts used in the practice of this invention are mixed metal oxides. The composition of the catalyst can vary widely and any of the catalysts known in the art for the oxidation of an alkane to an unsaturated aldehyde and/or carboxylic acid can be employed. Representative of these catalysts are those of Formula I

Mo₁V_{b}M¹_{c}M²_{c}Oₙ (I)

where M₁ is Te and/or Sb, M₂ is at least one of the elements from the group consisting of Nb, Ta, W, Ti, Al, Zr, Cr, Mn, Ga, Fe, Ru, Co, Rh, Ni, Pd, Pt, La, Bi, B, Ce, Sn, Zn, Si and In, b is from 0.01 to 1, c is from >0 to 1, d is from >0 to 1 and n is a number which is determined by the valences and frequency of the elements other than oxygen in (I). In one embodiment M¹ is Te and M² is Nb. The catalysts can be supported or unsupported, and they can be prepared by any one of a number of know procedures using known and commercially available equipment (see, for example, U.S. Pat. No. 6,180,825). Typical supports include silica, alumina, titania, aluminosilicate, diatomaceous earth and zirconium. The catalysts are typically in particulate form, e.g., granular, powder, pellet, bead, etc. Catalyst shape and catalyst particle size can vary to convenience.

Reactor

The process of this invention can be conducted in a fixed or fluidized bed reactor of any design. In one embodiment the process is conducted in a fluidized bed reactor. In one embodiment the process is conducted in a fixed bed reactor.

In one embodiment the reactor is a tube reactor of any configuration, e.g., straight, curved, serpentine, etc. While the cross-section of the tube is typically circular, it can be of any geometric shape, e.g., oval, polygonal, etc. Typically the cross-section of the tube is uniform along its length. If the cross-section of the tube is other than circular, then the widest length across the cross-section is the counterpart for the outer diameter of a tube with a circular cross-section.

The tubes can be made from any material that will maintain its integrity under reaction conditions of elevated temperature and pressure, and that is inert to the reaction starting materials, catalyst and reaction products under reaction conditions. Exemplary materials include metal (e.g., stainless steel), ceramic and glass. Tube wall thickness can also vary to convenience and on at least one level, it is a function of the material from which the tube is constructed.

Typically oxidation catalyst is tightly packed into the tube and held in place by a porous plug or stopper at or near each end of the tube. The plugs are porous to the starting gas mixture and/or the gaseous products of the oxidation reaction. The catalyst is packed in a manner that allows the starting gas mixture to flow over and around the catalyst particles under oxidation conditions so as to convert the propane to acrylic acid. Typically, a commercial process will employ more than one tube reactor at a time, and these are typically bundled into a single housing through which a heat transfer fluid is passed between and about the tubes to maintain a uniform temperature throughout the housing and in each tube. The reaction is exothermic and, as such, releases heat. The heat transfer fluid is used to remove heat and avoid the formation of hot spots which may adversely affect the catalyst. Suitable heat transfer media include inorganic salts and the DOWTHERM™ products. The reactor may consist of a single reactor stage, multiple reactor stages in separate reactor shells or multiple reactor stages in a single reactor shell. The optimum number of reactor stages is chosen to maximize the yield of AA while maintaining an economical capital and operating cost.

The invention is further described by the following examples. Unless indicated to the contrary, all parts and percentages are by weight.

### SPECIFIC EMBODIMENTS

The catalyst(s) used in these examples was a high-performance Mo/V/Te/Nb mixed metal oxide prepared according to the procedure described in U.S. Pat. No. 7,304,014. The process examples below are runs taken at similar conditions and compared at constant oxygen conversion.

### Example 1

An undiluted catalyst charge (4.0 cc) is loaded into a 0.25 inch OD 316 stainless steel (SS) tube that is encased by a 1-inch diameter brass jacket. The jacket facilitates temperature control of the process and its isothermal operation. The feed composition in weight percent is 6.0% propane, 11.3% oxygen, 40% steam, with nitrogen as the balance. Residence time is 3.0 seconds at atmospheric pressure. Reactor temperatures are adjusted to give the desired conversion. Gas and liquid products are analyzed by gas chromotography (GC).

### Example 2

Example 1 is repeated except that the feed composition is 6.0 wt % propane, 11.3 wt % oxygen, 20 wt % steam, with nitrogen as the balance.

### Example 3

Example 1 is repeated except that the feed composition is 6.0 wt % propane, 11.3 wt % oxygen, 10 wt % steam, with nitrogen as the balance.

### Example 4

Example 1 is repeated except that the feed composition is 6.0 wt % propane, 11.3 wt % oxygen, 5 wt % steam, with nitrogen as the balance.

The results of the four runs are reported in the following table. The data clearly shows that a reduction in steam to less than 20 wt % results in a reduction in PA production all else being the same.

TABLE
Reaction Conditions and Results of Examples 1-4

| | Steam | | C₃ | O₂ | AA | |
|---|---|---|---|---|---|---|
| | level, | Temp, | conv, | conv, | yield, | PA, |
| | % | ° C. | % | % | % | ppm |
| Example 1 | 40 | 350 | 59.0 | 73.4 | 41.0 | 1700 |
| Example 2 | 20 | 355 | 60.0 | 77.0 | 42.4 | 950 |
| Example 3 | 10 | 360 | 62.5 | 80.0 | 42.4 | 470 |
| Example 4 | 5 | 360 | 59.9 | 77.8 | 41.1 | 330 |

Although the invention has been described with certain detail through the preceding description of the preferred embodiments, this detail is for the primary purpose of illustration. Many variations and modifications can be made by one skilled in the art without departing from the spirit and scope of the invention as described in the following claims.

1. A process for the direct oxidation of propane to acrylic acid, the process comprising the step of contacting under oxidation conditions a gaseous mixture comprising, in weight percent (wt %) based on the weight of the mixture, 5-10% propane, 5-15% oxygen, 1-20% steam and the balance a diluent gas, with a propane oxidation catalyst.

2. The process of claim 1 in which the propane comprises 1 or more wt % propylene based on the weight of the propane.

3. The process of claim 1 in which the propane comprises less than 1 wt % propylene based on the weight of the propane.

4. The process of claim 1 in which the steam is present at 10-15 wt % based on the weight of the gaseous mixture.

5. The process of claim 1 in which the diluent gas is nitrogen.

6. The process of claim 1 in which the oxidation catalyst is of Formula I
Mo₁V_{b}M¹_{c}M²_{c}Oₙ (I)
where M₁ is Te and/or Sb, M₂ is at least one of the elements from the group consisting of Nb, Ta, W, Ti, Al, Zr, Cr, Mn, Ga, Fe, Ru, Co, Rh, Ni, Pd, Pt, La, Bi, B, Ce, Sn, Zn, Si and In, b is from 0.01 to 1, c is from >0 to 1, d is from >0 to 1 and n is a number which is determined by the valences and frequency of the elements other than oxygen in (I).

7. The process of claim 1 conducted in a tube reactor.

8. The process of claim 7 in which the tube reactor is a fixed-bed, multi-tube reactor.

9. The process of claim 7 in which the reactor comprises one reactor stage in one reactor shell, or more than one reactor stage in multiple reactor shells, or multiple reactor stages in one reactor shell.

10. The process of claim 7 in which the reactor is made of stainless steel and has a circular cross-section.

11. The process of claim 1 in which the oxidation conditions include a pressure from 0 to 100 psig (0 to 0.70 MPa), a reaction temperature from 0 to 550° C., a gas space velocity of 100 to 10,000 hr⁻¹, and a residence time from 0.1 to 10 seconds.
